(19) United States
(12) Patent Application Publication
(10) Pub. No.: US 2016/0095681 A1
(43) Pub. Date: Apr. 7, 2016

(54) SONIC TOOTHBRUSH AND WHITENING COMPOUND

(71) Applicant: ISHINE LLC, FRANKLIN SQUARE, NY (US)

(72) Inventor: ERIC WEINSTEIN, OLD BROOKVILLE, NY (US)

(21) Appl. No.: 14/505,911

(22) Filed: Oct. 3, 2014

(51) Int. Cl. A61C 17/34; A61C 17/22; A61C 1/08; A61C 19/06; A61N 5/06; A46B 15/00; A46B 11/00

(52) U.S. Cl. CPC A61C 17/3481; A46B 15/0034; A46B 15/0081; A61C 17/224; A46B 11/0072; A61C 19/066; A61N 5/062; A61C 1/088; A61N 2005/0658; A61N 2005/0651

(57) ABSTRACT

Technologies are generally described for systems, methods and devices relating to sonic toothbrush and whitening compound. In some examples, systems may comprise a sonic toothbrush. The sonic toothbrush may include a head section and a handle section. The head section may include a head at a first end and a connector attached to the handle section at a second end. The head may include bristles, a blue light emitting diode (LED) and a white LED. The handle section may include a sonicator configured to vibrate the head. The handle section may include controls to provide power to the sonicator, to select a speed of the sonicator, to provide power to the blue LED, and to provide power to the white LED. The systems may comprise a whitening compound including hydrogen peroxide. The hydrogen peroxide in the whitening compound may be activated when exposed to light emitted by the blue LED.

### BACKGROUND

Oral hygiene may be maintained through the use of oral hygiene devices and products. Oral hygiene devices and products include chew sticks, toothbrushes, floss, mouth washes and toothpastes. There is great variety within each product or device group. Oral hygiene products and devices may be use together or independently.

### SUMMARY OF THE INVENTION

In one example, oral cleaning and tooth whitening systems are generally described. The systems may comprise a sonic toothbrush. The sonic toothbrush may include a head section and a handle section. The head section may include a head at a first end and a connector at a second end. The connector may be attached to the handle section. The head may include a first side and a second side. The first side may include bristles, a blue light emitting diode (LED) and a white LED. The second side may include a tongue cleaner. The handle section may include a sonicator. The sonicator may be configured to vibrate the head. The handle section may include a control to provide power to the sonicator, a control to select a speed of the sonicator, a control to provide power to the blue LED, and a control to provide power to the white LED. The systems may comprise a whitening compound. The whitening compound may include hydrogen peroxide. The hydrogen peroxide in the whitening compound may be activated when exposed to blue light emitted by the blue LED.

In another example, methods for whitening teeth are generally described. The methods may comprise placing a whitening compound on to bristles on a head of a sonic toothbrush. The methods may comprise pressing a power control button on a handle of the sonic toothbrush to power the sonic toothbrush. The powering may cause vibrations of the head. The methods may comprise selecting a speed setting. The methods may comprise pressing a control button on the handle of the sonic toothbrush to power a blue LED to emit blue light. The methods may comprise applying the whitening compound on the bristles to a tooth such that the vibrations of the head of the sonic toothbrush disperses the whitening compound upon the tooth while exposing the whitening compound to the emitted blue light.

In another example, oral cleaning and tooth whitening devices are generally described. The devices may comprise a sonic toothbrush. The sonic toothbrush may include a head section and a handle section. The head section may include a head at a first end and a connector at a second end. The connector may be attached to the handle section. The head may include a first side and a second side. The first side may include bristles, a blue light emitting diode (LED), and a white LED. The second side may include a tongue cleaner. The handle section may include a sonicator. The sonicator may be configured to vibrate the head. The handle section may include a control to provide power to the sonicator, a control to select a speed of the sonicator, a control to provide power to the blue LED, and a control to provide power to the white LED. The devices, when powered and the blue LED powered, configured to apply a whitening compound, including hydrogen peroxide, with the bristles to a tooth such that the vibration of the head of the sonic toothbrush disperses the whitening compound upon the tooth while exposing the whitening compound to blue light emitted by the blue LED, wherein the hydrogen peroxide in the whitening compound is activated by the blue light.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims taken in conjunction with the accompanying drawings. Understanding that these drawings depict only some embodiments in accordance with the disclosure and are therefore not to be considered limiting of its scope, the disclosure will be described with additional specificity and detail by reference to the accompanying drawings in which:

FIG. 1 is a front view of a toothbrush;

FIG. 2 is a rear view of a toothbrush;

FIG. 3 is a side view of a toothbrush;

FIG. 4 is a front view of a handle section illustrating control buttons of a toothbrush;

FIG. 5 is a front view of a toothbrush head illustrating the light and arrangement of bristles;

FIG. 6 is a side view of a toothbrush head illustrating arrangement and height of bristles; and

FIG. 7 is a side view of a charger base for a toothbrush;

all in accordance with an embodiment of the invention.

### DETAILED DESCRIPTION

In the following detailed description, reference is made to the accompanying drawings which form a part thereof. In the drawings, similar symbols typically identify similar components unless context indicates otherwise. The illustrative embodiments described in the detailed description, drawings and claims are not meant to be limiting. Other embodiments may be utilized and other changes may be made without departing from the spirit or scope of the subject matter presented herein. It will be readily understood that the aspects of the present disclosure as generally described herein and as illustrated in the accompanying figures can be arranged, substituted, combined, separated and/or designed in a wide variety of different configurations all of which are explicitly contemplated herein.

Briefly stated, technologies are generally described for systems, methods and devices relating to sonic toothbrush and whitening compound. In some examples, systems may comprise a sonic toothbrush. The sonic toothbrush may include a head section and a handle section. The head section may include a head at a first end and a connector at a second end. The connector may be attached to the handle section. The head may include a first side and a second side. The first side may include bristles, a blue light emitting diode (LED) and a white LED. The second side may include a tongue cleaner. The handle section may include a sonicator. The sonicator may be configured to vibrate the head. The handle section may include a control to provide power to the sonicator, a control to select a speed of the sonicator, a control to provide power to the blue LED, and a control to provide power to the white LED. The systems may comprise a whitening compound. The whitening compound may include hydrogen peroxide. The hydrogen peroxide in the whitening compound may be activated when exposed to blue light emitted by the blue LED.

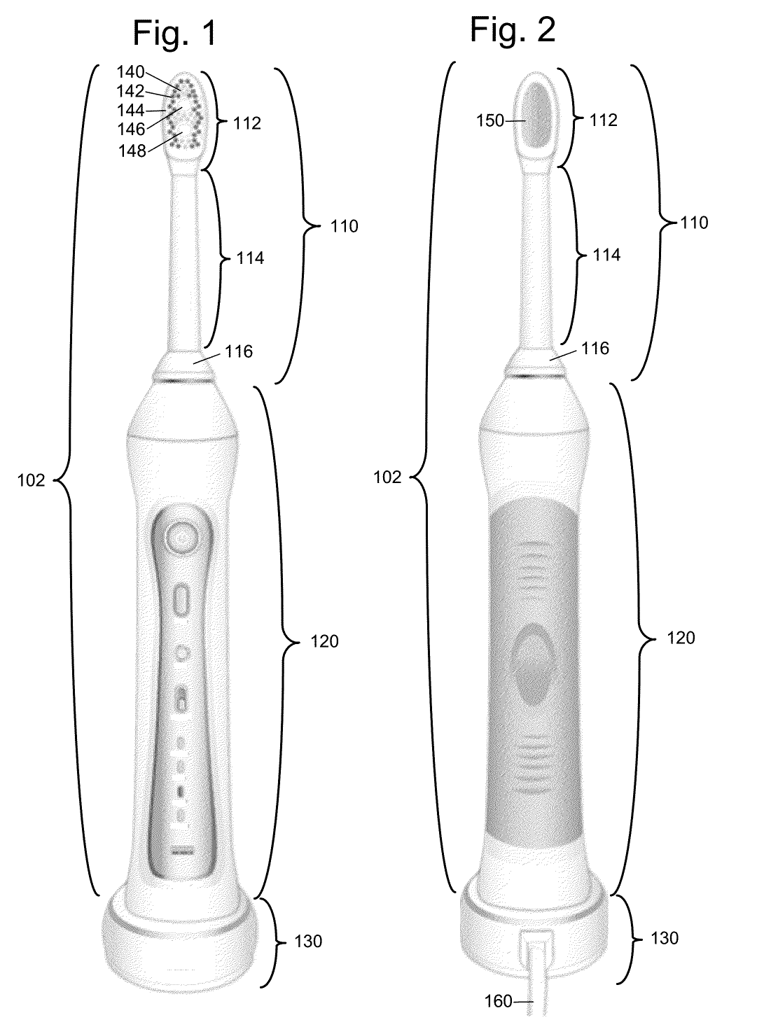

FIG. 1 is a front view of a toothbrush in accordance with an embodiment of the invention. A sonic toothbrush 102 may include a head section 110 and a handle section 120. Head section 110 may include a head 112 at a first end, a connector 116 at a second end, and an arm extension 114 extending between head 112 and connector 116. A front of head 112 may include bristles 140, 142, 144 and lights 146, 148. Connector 116 may attach head section 110 to handle section 120. Front of handle section 120 may include buttons and controls to operate sonic toothbrush 102. Handle section 120 may sit in a charger base 130 when not in use. Charger base 130 may secure sonic toothbrush 102 in an upright position as well as provide charging to sonic toothbrush 102.

FIG. 2 is a rear view of a toothbrush in accordance with an embodiment of the invention. Those components in FIG. 2 that are labeled identically to components of FIG. 1 will not be described again for the purposes of clarity. A rear of head 112 may include a tongue cleaner 150. Tongue cleaner 150 may include raised bumps and may exfoliate and clean when scrubbed against an oral surface such as a tongue. The rear of handle section 120 may include a grip which may improve a grip of a user of sonic toothbrush 102. Rear of charger base 130 may include a cord 160. Cord 160 may extend to a standard alternating current (A/C) plug which may be plugged into an electrical outlet to provide power to charge batteries within sonic toothbrush 102.

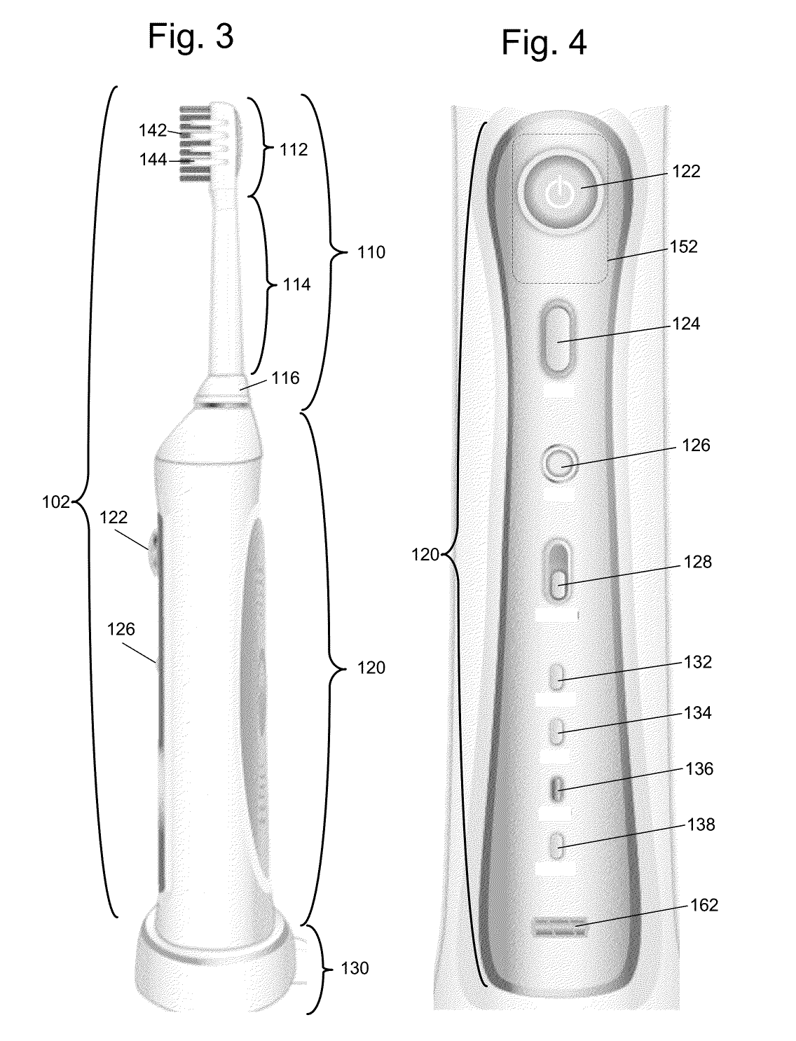

FIG. 3 is a side view of sonic toothbrush 102 in accordance with an embodiment of the invention. Those components in FIG. 3 that are labeled identically to components of FIG. 1-2 will not be described again for the purpose of clarity. Handle portion 120 of sonic toothbrush 102 may include a grip along a rear of handle section 120. Handle portion 120 may include buttons and controls for operation of sonic toothbrush 102 along a front of handle section 120. Power button 122 to provide power to sonic toothbrush 102 and shine button 126 to provide power to light 148 may protrude out from front of handle section 120.

FIG. 4 is a front view of handle section 120 illustrating control buttons of sonic toothbrush 102 in accordance with an embodiment of the invention. Those components in FIG. 4 that are labeled identically to components of FIG. 1-3 will not be described again for the purposes of clarity. Front of handle section 120 may include power button 122, a speed control 124, shine button 126, a whitening switch 128, a sensitive setting indicator 132, a gentle setting indicator 134, an intense setting indicator 136, a polishing setting indicator 138, and a toothbrush battery charge indicator 162. Power button 122 may be used to power on sonic toothbrush 102.

Sonic toothbrush 102 may be a sonic toothbrush and may include a sonicator 152 within handle 120. Sonicator 152 inside handle 120 may vibrate head 112 when powered. Sonic toothbrush 102 may be powered by a battery or batteries in handle section 120. Sonic toothbrush 102 may be powered by a rechargeable battery in handle section 120. Sonicator 152 may operate at a frequency range of 20,000 Hz to 1.6 MHz. Speed control 124 may be used to set the frequency of sonicator 152, and thus the speed of vibration of head 112, depending on a user preference or the operation (type of cleaning) to be performed by sonic toothbrush 102. Indicators 132, 134, 136, or 138 may become illuminated or light up when the indicated speed setting of sonicator 152 is selected with speed control 124. For example, sensitive setting indicator 132, gentle setting indicator 134, intense setting indicator 136, or polishing setting indicator 138 may light up in response to a selection of the respective speed setting by speed control 124. Shine button 126 may be used by a user of sonic toothbrush 102 to power on light 148. Whitening switch 128 may be used by a user of sonic toothbrush 102 to power on light 146. Battery charge indicator 162 may illustrate a battery and may be illuminated to indicate an amount of charge remaining. Battery charge indicator 162 may indicate an amount of charge contained in the battery of sonic toothbrush 102 by lighting a fraction of the illustrated battery related to the amount of charge contained in the battery compared to a fully charged battery. For example, if the battery contained a full charge, the whole battery in battery indicator 162 may be illuminated. In another example, if the battery contained half of a full charge, half of the battery in battery indicator 162 may be illuminated.

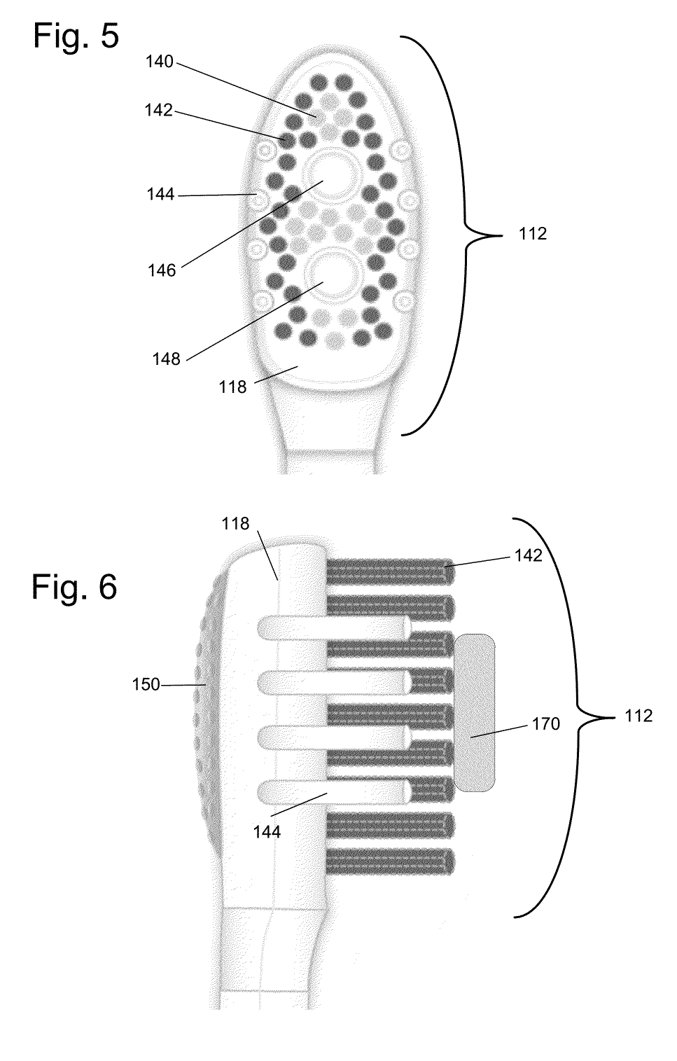

FIG. 5 is a front view of a toothbrush head 112 illustrating lights 146, 148 and arrangement of bristles 140, 142, 144 in accordance with an embodiment of the invention. FIG. 6 is a side view of a toothbrush head 112 illustrating arrangement and height of bristle 140, 142, 144 in accordance with an embodiment of the invention. Those components in FIG. 5 and FIG. 6 that are labeled identically to components of FIG. 1-4 will not be described again for the purposes of clarity. Head 112 may include bristles 140, 142 and 144. Bristles 140 and 142 may extent perpendicularly from head base 118. Bristles 140 may clean the top of a user's tooth such as a biting or chew surface when sonic toothbrush 102 is used. Bristles 142 may be longer in length than bristles 140 and may clean the sides or front and back of a user's tooth when sonic toothbrush 102 is used. Bristles 144 may be wider in diameter than bristles 140 and 142, and may be positioned on the outer edges of head base 118. Bristles 144 may clean and massage the gum line of a user when sonic toothbrush 102 is used.

Lights 146 and 148 may be within head base 118. Lights 146, 148 may be light-emitting diode (LED) lights. Light 146 may emit violet/blue light in a visible light spectrum from 400 nanometers to 700 nanometers. Blue light emitted by light 146 may provide antibacterial action within the mouth or oral cavity of a user of sonic toothbrush 102. Light 148 may emit white light in a visible light spectrum. Shine button 126 on handle section 120 (as shown in FIG. 4) may be used to complete a circuit so that power may be sent from a battery of sonic toothbrush 102 to light 148. Whitening switch 128 on handle section 120 (as shown in FIG. 4) may be used to complete a circuit so that power may be sent from a battery of sonic toothbrush 102 to light 146. Powered lights 146, 148 may emit light when powered.

Sonic toothbrush 102 may be used with a tooth whitening compound 170. Tooth whitening compound 170 may be a tooth whitening gel. Tooth whitening compound 170 may whiten teeth when brushed onto the surface of teeth by sonic toothbrush 102. Tooth whitening compound 170 may be activated by exposure to blue light emitted by light 146 in sonic toothbrush 102. Tooth whitening compound 170 may include hydrogen peroxide. Hydrogen peroxide in whitening compound 170 may be 5-38% by mass. Hydrogen peroxide in tooth whitening gel 170 may be activated by blue light emitted by light 146. White light 148 on sonic toothbrush 102 may be used to enhance vision within the mouth of a user of sonic toothbrush 102. White light 148 may provide enhanced visibility and allow a user of sonic toothbrush 102 to perform an oral evaluation for disease, oral cancer, or infections. White light 148 may illuminate the oral cavity to enhance visibility to provide a user of sonic toothbrush 102 better ability to perform oral hygiene, evaluate dental care and improve oral hygiene care.

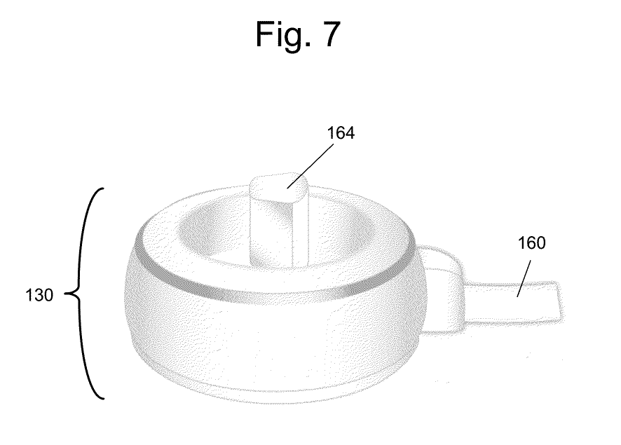

FIG. 7 is a side view of a charger base for a toothbrush in accordance with an embodiment of the invention. Those components in FIG. 7 that are labeled identically to components of FIG. 1-6 will not be described again for the purposes of clarity. Charger base 130 may include a cord 160 and a docking support 164. Docking support 164 and walls of charger base 130 may provide a docking station for sonic toothbrush 102. Cord 160 may be connected to a power source such as an electric outlet. Power from cord 160 may be transferred to charge batteries in sonic toothbrush 102 when sonic toothbrush 102 is docked in charger base 130 and attached to docking station 164.

In an example, a user of sonic toothbrush 102 may want to maintain oral hygiene and whiten teeth. A user of sonic toothbrush 102 may observe charge indicator 162 on sonic toothbrush 102 when sonic toothbrush 102 is docked in charger base 130 and cord 160 is attached to an electric outlet. The user may undock sonic toothbrush 102 from charger base 130 when charge indicator 162 indicates that the battery of sonic toothbrush 102 has sufficient charge.

In another example, a user may want whiter teeth. The user may place a sufficient quantity of tooth whitening compound 170 on bristles 140, 142, and 144 of sonic toothbrush 102. The user may press power control button 122 on handle section 120 of sonic toothbrush 102 to power sonicator 152 in handle 120. Powering of sonicator 152 may cause movement of head 112. The user may select a speed setting by pressing speed control 124 until the desired speed is indicated by a speed indicator lighting up. The user may switch whitening switch 128 on handle section 120 of the sonic toothbrush 102 to power blue LED 146 to emit blue light. The user may apply tooth whitening compound 170 on bristles 140, 142, and 144 to a tooth such that the vibration of head 112 disperses tooth whitening compound 170 upon the tooth. Tooth whitening compound 170 may be exposed to blue light emitted by blue LED 146 while being dispersed upon the tooth.

Among other potential benefits, a device in accordance with the disclosure may whiten a user's teeth in the convenience of their own home. Tooth whitening may not require trays and strips and may be performed as part of a routine oral hygiene practices. White light may be used to inspect a user's mouth for oral disease or cancer as well as to inspect whitening progress. Tooth whitening may be superior to other over the counter methods and may be of similar quality to a dental office treatment.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An oral cleaning and tooth whitening system, the system comprising:
a sonic toothbrush, wherein the sonic toothbrush includes a head section and a handle section, where the head section includes a head at a first end and a connector at a second end, wherein the connector is attached to the handle section, where the head includes:
a first side and a second side, the first side includes:
bristles;
a blue light emitting diode (LED);
and a white LED;
the second side includes:
a tongue cleaner;
the handle section includes:
a sonicator, wherein the sonicator is configured to vibrate the head;
a control to provide power to the sonicator;
a control to select a speed of the sonicator;
a control to provide power to the blue LED;
a control to provide power to the white LED;
a whitening compound, wherein the whitening compound includes hydrogen peroxide and the hydrogen peroxide in the whitening compound is activated when exposed to blue light emitted by the blue LED.

2. The system of claim 1, wherein the sonic toothbrush includes a rechargeable battery, the system further comprising a charger base, wherein the charger base is configured to provide a docking station for the sonic toothbrush and transfer power to charge the rechargeable battery.

3. The system of claim 2, wherein the handle section further includes a battery charge indicator.

4. The system of claim 1, wherein the whitening compound includes 5-38% hydrogen peroxide by mass.

5. The system of claim 1, wherein the control to select a speed of sonicator 152 is configured to select a speed setting of sonicator 152 from one of sensitive, gentle, intense, and polishing and wherein the handle section of the sonic toothbrush includes speed setting indicators including speed setting indicators of sensitive, gentle, intense, and polishing speed settings.

6. The system of claim 5, wherein the speed setting indicators of sensitive, gentle, intense, and polishing speed settings light up upon selection of the respective setting by the control to select a speed.

7. The system of claim 1, wherein the white LED is configured to illuminate and enhance visibility in an oral cavity.

8. The system of claim 1, wherein the bristles include bristles of different lengths and widths.

9. The system of claim 1, wherein:
the sonic toothbrush includes a rechargeable battery;
the handle section includes a battery charge indicator;
the whitening compound includes 5-38% hydrogen peroxide by mass;
the control to select a speed of sonicator 152 is configured to select a speed setting of sonicator 152 from one of sensitive, gentle, intense, and polishing;
the handle section of the sonic toothbrush includes indicators for sensitive, gentle, intense, and polishing speed settings; and
the indicators for sensitive, gentle, intense, and polishing speed settings light up upon selection of the respective setting by the control to select a speed.

10. A method for whitening teeth, the method comprising:
placing a whitening compound on to bristles on a head of a sonic toothbrush;
pressing a power control button on a handle of the sonic toothbrush to power the sonic toothbrush, wherein the powering causes vibrations of the head;
selecting a speed setting;
pressing a control button on the handle of the sonic toothbrush to power a blue LED to emit blue light;
and
applying the whitening compound on the bristles to a tooth such that the vibrations of the head of the sonic toothbrush disperses the whitening compound upon the tooth while exposing the whitening compound to the emitted blue light.

11. The method of claim 10, wherein the sonic toothbrush includes a rechargeable battery, the method further comprising charging the sonic toothbrush with a charger base, wherein the charger base is configured to provide a docking station for the sonic toothbrush and provide power to charge the rechargeable battery.

12. The method of claim 11, wherein the charger base includes a cord and the cord is configured to connect to an electric outlet.

13. The method of claim 10, wherein the handle section further includes a battery charge indicator.

14. The method of claim 10, wherein the speed settings including, sensitive, gentle, intense, and polishing.

15. The method of claim 14, wherein the handle section of the sonic toothbrush includes indicators for sensitive, gentle, intense, and polishing speed settings.

16. The method of claim 15, wherein the indicators for sensitive, gentle, intense, and polishing speed settings light up upon selection of the respective setting.

17. The method of claim 10, further comprising, after applying the whitening compound on the bristles to the tooth, pressing the control button on the handle of the sonic toothbrush to power a white LED to illuminate and enhance visibility in an oral cavity.

18. The method of claim 10, wherein the bristles include bristles of different lengths and widths.

19. An oral cleaning and tooth whitening device, the device comprising:
a sonic toothbrush, wherein the sonic toothbrush includes:
a head section and a handle section, where the head section includes a head at a first end and a connector at a second end, wherein the connector is attached to the handle section, where the head includes:
a first side and a second side, the first side includes:
bristles;
a blue light emitting diode (LED); and
a white LED;
the second side includes:
a tongue cleaner;
the handle section includes:
a sonicator, wherein the sonicator is configured to vibrate the head;
a control to provide power to the sonicator;
a control to select a speed of the sonicator;
a control to provide power to the blue LED;
a control to provide power to the white LED;
the device, when powered and the blue LED powered, configured to apply a whitening compound, including hydrogen peroxide, with the bristles to a tooth such that the vibration of the head of the sonic toothbrush disperses the whitening compound upon the tooth while exposing the whitening compound to blue light emitted by the blue LED, wherein the hydrogen peroxide in the whitening compound is activated by the blue light.

20. The device of claim 19, wherein the whitening compound includes 5-38% hydrogen peroxide by mass.
